(19) United States
(12) Patent Application Publication
(10) Pub. No.: US 2005/0027303 A1
(43) Pub. Date: Feb. 3, 2005

(54) Pelvic waypoint clamp assembly and method

(76) Inventors: David R. Lionberger, Houston, TX (US); Stephanie Lionberger, Houston, TX (US)

Correspondence Address: HAHN LOESER & PARKS, LLP, One GOJO Plaza, Suite 300, AKRON, OH, 44311-1076, US

(21) Appl. No.: 10/870,322

(22) Filed: Jun. 17, 2004

Related U.S. Application Data
(60) Provisional application No. 60/479,144, filed on Jun. 17, 2003.

(51) Int. Cl. A61B 17/58

(52) U.S. Cl. 606/102

(57) ABSTRACT

A pelvic waypoint clamp assembly designed for external attachment to a patient's hips. The clamp assembly serves as a foundation for a fixation mount (i.e. a passive or active tracker) that, when in communication with conventional computer-assisted orientation systems, is used to determine, non-invasively, the center of the femoral head prior to or during knee arthroscopy.

This is application claims the benefit of the filing of co-pending U.S. provisional application Ser. No. 60/479,144, filed Jun. 17, 2003, and which is incorporated by reference herein in its entirety.

### BACKGROUND AND SUMMARY OF THE INVENTION

Through the use of external computer-assisted orientation systems, the brevity and the accuracy of a routine total knee replacement can be brought to a totally new level. Quite simply, the accuracy of the use of the external orientation system affords the ability of a totally new instrumented surgical procedure.

Crucial to the accuracy of a computer assisted orientation system for total or unicondylar knee replacement is the insertion of a fixation pin into pelvic bone to establish the center of the femoral head (i.e. set the “waypoint” on the pelvis). More specifically, an incision is made on the most anterior aspect of the iliac crest, afterwhich a pin guide is inserted into a hole drilled previously drilled in the bone at the desired location for the pin guide.

A complex array of calculations are utilized to describe the center of the femoral head using data generated about the location of the fixation pin. By establishing the center of the femoral head, the mechanical axis or weight bearing line can be established in order to properly insert the total knee replacement implants. This then is programmed into the orientation system which the computer assisted orientation system utilizes to accommodate and accurately calculate this orientation.

The current methods of setting the waypoint described above, namely, the invasive insertion of a fixation pin into the patient's pelvis, has many disadvantages. Specifically, this method carries with it risks of infection and injury to intra-pelvic structures if the pin were to impale the viscera of the abdominal region. This invasive method also adds a significant morbidity to the patient's recovery.

To overcome these problems, the inventors have developed an external device, and thus, non-invasive pelvic stabilizing waypoint clamping device that provides a rigid, yet reproducible substitution for prior art fixation methods without the use of an open incision or exposure to the pelvis. The device of the present invention comprises a pelvic stabilizing clamp assembly configured to rigidly adhere to the crest of the pelvis and provide a movement with the pelvis synchronously while applying range of motion to the hip joint. By this method, the center of the hip joint (i.e. center of the femoral head 100) is established by the routine methods described in the protocol for the computer assisted orientation system; however, without the added morbidity of the incision point. The CAOS software is not changed, but merely obtains its data from a different point to make the desired calculations.

More specifically, the present invention, in certain aspects, is directed to a clamp assembly for attachment to a patient's hip. The clamp assembly comprises a pair of opposing jaws, with each of the jaws having a base secured to an elongated rod. The clamp assembly further includes a waypoint fixation mount secured to the rod between a pair of opposing jaws, the waypoint fixation mount being designed for attachment to a computer assisted orientation system, and wherein the system is programmed to generate data corresponding to a position of the fixation device for use in knee arthroscopy. The computer assisted orientation system may be further programmed, through a series of algorithmic calculations, to establish of the center of the femoral head prior to or during the knee arthroscopy. In certain aspects of the invention, at least one of the bases of the jaws is movable horizontally along a horizontal axis parallel with the elongated rod, such that the distance between the jaws may be adjusted upon the rod. The jaws may also be adjustable vertically along a vertical axis perpendicular to the rod. Finally, with respect to adjustability of the clamp assembly to accommodate patients of different sizes, at least one of the jaws may be configured to rotate about a vertical axis perpendicular to the elongated rod. The jaws may comprises a solid plate or a loop secured to a base. Preferably, the jaws are cup-like in configuration in order to better conform to the shape of the exterior hip of the patient.

The present invention is also directed to a method of determining the center of the femoral head and comprises (1) attaching a waypoint fixation device (preferably the pelvic clamp of the present invention) externally to a patient, the device being positioned on the anterior side of a patient; (2) placing the waypoint fixation device in communication with a computer assisted orientation system; (3) recording and generating data with respect to the location of the fixation device via the computer assisted orientation system; and (4) calculating the center of hip joint (i.e. center of the femoral head) based in part upon the data generated.

### BRIEF DESCRIPTION OF THE FIGURES

FIG. 1 is a one perspective view of the inventive clamp assembly.

FIG. 2 is a second perspective view of the inventive clamp assembly illustrated in FIG. 1.

FIG. 3 is a schematic anterior view of the inventive clamp assembly secured to a patient's hips (the hips shown in skeletal form).

### DETAILED DESCRIPTION OF THE INVENTION

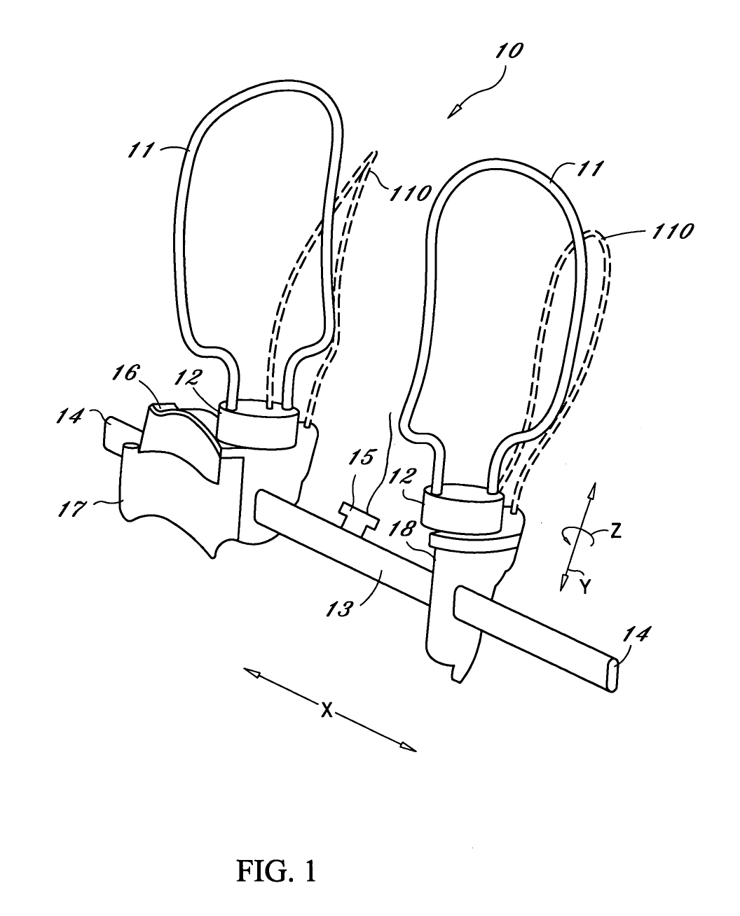

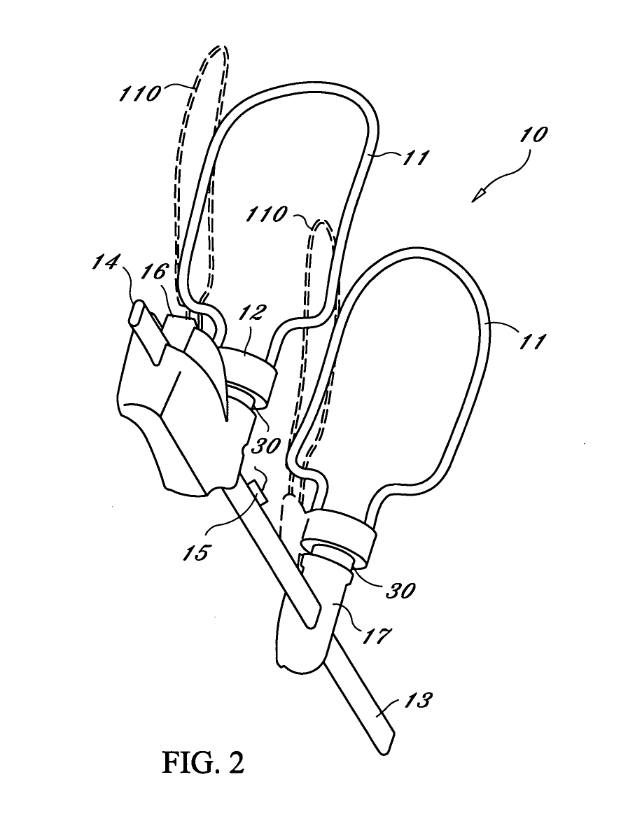

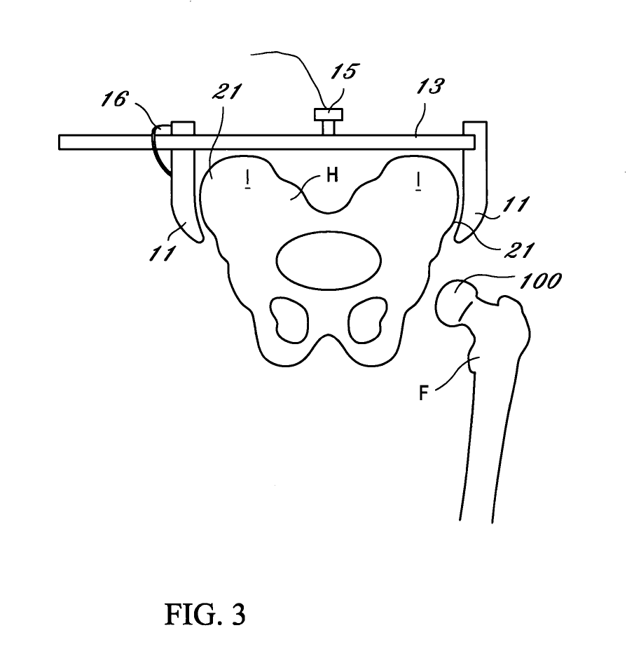

Referring now to the figures, the present invention is directed to a clamp assembly 10 designed for external attachment to a patient's hips (along the iliac crest I) prior to and/or during knee arthroscopy. FIG. 3 illustrates a sketch of the clamp assembly secured to the hip H, with the iliac crest referenced generally as I, the femur generally as F, and the femoral head at 100. The clamp assembly comprises a pair of opposing jaws 11, each of the jaws having a base 17, 18. The base, in turn, is secured to an elongated rod 13. The clamp assembly includes one or more of three features that provide for adjustability in size of the clamp assembly in order to accommodate a variety of different size patients. One such feature is the ability of the jaws to rotate about axis Y, as shown in FIG. 1, for example. In this aspect of the invention, the base may be provided with a swivel platform 12 to which the jaws are directly attached. The swivel platform 12 allows the jaws to rotate, preferably up to 360°, about vertical axis Y in direction Z, and then lock into place. The jaws 110 shown in phantom in FIGS. 1-2 illustrate different positions of the jaws via the rotating feature of the instant invention. One or both jaws 11 may be provided with a means to rotate about vertical axis Y. It will also be appreciated by the skilled artisan that the structure and means by which the jaws may rotate about axis Y (in direction Z) may be varied without departing from the scope and spirit of the present invention.

A second feature of the invention that provides for size adjustability of the clamp assembly is the ability to adjust or move one or both of the jaws horizontally along a horizontal axis X that is aligned parallel with the elongated rod 13, as shown in FIG. 1, for example. This feature allows the clamp assembly to be lengthened or shortened about axis X in order accommodate the hip width of a particular patient. In one embodiment, the base 17 has an opening through which the rod is engaged. As shown in FIG. 1, one base 17 may be adjustable in such a manner while the second base 18 is fixed upon the rod. Base 17 includes a release lever 16 that, when depressed, allows the jaw to move horizontally toward ends 14 of the rod, and when released, lock the base into place at the desired position on the rod. Alternatively, each of the two bases may be movable, and if desired, designed similarly to base 17. It will be appreciated by those of ordinary skill in the art, however, that the base 17, 18 may be modified, and thus differ from the design illustrated in the figures, and yet remain within the scope of present invention for both the fixed base 18 and adjustable base 17 aspects of this feature of the invention.

A third feature of the invention that provides for size adjustability of the clamp assembly is the provision of a telescoping means within the base that allows for the jaws to be moved vertically along axis Y, thereby allowing for the adjustment of the height of each jaw relative the base or elongated rod. The telescoping aspect of the clamp assembly may be provided by a rod within a sleeve, referenced generally as 30 in FIG. 2. It will be appreciated by those of ordinary skill in the art, however, that any means (telescoping or otherwise) for providing height adjustability of the jaws in the vertical direction (i.e. along axis Y) may be employed without departing from the scope of the present invention.

The shape of the individual jaws 11 may vary as desired; however, a preferred cup-like configuration is illustrated in the figures. The jaws may comprise a solid plate (not shown), cup-like or flat, or the jaws may comprise a loop with an opening, as shown in the figures, for better accommodating and securing the sides 21 of the patient's hips.

The clamp assembly 10 further includes a waypoint fixation mount 15 secured to the rod 13 and centrally positioned thereon. The mount 15 is a conventional fixation tracker (passive or active) known by those ordinary skill in the art that is designed for attachment to or communication with a computer assisted orientation system (not shown) that includes software programmed to generate data corresponding to the position of the fixation mount 15 in surgery, primarily in knee arthroscopy. It will be appreciated by the skilled artisan that the fixation mount 15 illustrated herein is merely a schematic representation, and thus, other fixation mount designs may be employed. It will also be recognized by those of ordinary skill in the art that the fixation mount may be positioned at different locations along the clamp assembly (i.e. other than that depicted in the figures herein). Conventional computer assisted orientation systems, such as the KNEE NAVIGATION V1.1 system, vended by Stryker (Kalamazoo, Mich.), may be employed with the present invention. Specifically, the computer assisted orientation system is programmed, through a series of algorithmic calculations, to establish the center of the femoral head 100. In operation, the pelvic clamping device 10 is secured anteriorly to the patient's hips and remains rigidly adhered to the crest of the pelvis (externally) while allowing unrestricted range of motion of the hip. Preferably, the clamping assembly is stabilized on the patient's body via a belt (not shown) or similar type of support apparatus worn by the patient. By this method, the center of the femoral head 100 is established by the routine methods described in the protocol for conventional computer assisted orientation systems without the added morbidity of the incision point required in the prior art. This is achieved with no modifications being necessary to the navigational software—that is, the inventive pelvic clamping device may be directly substituted for the conventional internal fixation pins known and currently employed in the art. As described in Example 1, the experimental results show that the inventive pelvic clamp assembly and method provide a less invasive and less dangerous method for accurately establishing the center of the femoral head.

### EXAMPLE

A study was performed using 10 patients receiving either total or unicondylar knee replacement guided with a Surgical Navigation system, which utilized a pelvic fixation pin drilled into the pelvic crest. After the procedure was completed, the inventive pelvic clamp was applied and additional measurements were taken to compare the accuracy of the two techniques in localizing the femoral head center. With both pelvic identifiers, the femur, with an infrared tracker, was manipulated to generate points approximating the surface of a sphere used to determine the femoral head center. The measurements consisted of the root mean square error of each instantaneous head center location.

The average RMS error of the femoral head center using the fixed pelvic pin was 4.5+/−0.5 cm versus 5.4+/−0.5 cm for the pelvic clamp. There was no statistical difference between the accuracy of the two techniques (p=0.24). In addition, there was no significant difference in the standard deviation of head center positions achieved using the two techniques (pin: 1.58 cm, clamp: 1.49 cm; F=0.885, p=0.86). The average distance calculated from the center of the head to the femoral tracker was 419 cm. Thus, the difference in the error expected in the calculation of the mechanical axis between the two techniques is 0.1°. This error would have the effect of changing the angle of the femoral component by +/−8 minutes.

1. A clamp assembly for attachment to a patient's hip in knee arthroscopy, said assembly comprising:
a. a pair of opposing jaws secured to an elongated rod, each of said jaws configured to attach externally to a patient's hip;
b. a waypoint fixation mount secured to said rod and positioned between said pair of opposing jaws, said waypoint fixation mount designed for attachment to a computer assisted orientation system, and wherein said system is programmed to generate data corresponding to a position of said fixation mount for use in said knee arthroscopy.

2. The clamp assembly of claim 1, wherein said computer assisted orientation system is further programmed, through a series of algorithmic calculations, to establish the center of a femoral head of said patient prior to or during said knee arthroscopy.

3. The clamp assembly of claim 1, said pair of opposing jaws comprising a first jaw and a second jaw, and wherein said first jaw is secured to a base, said base movably secured to said elongated rod, such that said base, in combination with said first jaw, may be moved horizontally a desired distance away from said second jaw in order accommodate a width of said patient's hip.

4. The clamp assembly of claim 3, wherein said second jaw is secured to a base, said second jaw base secured to said elongated rod.

5. The clamp assembly of claim 4, wherein said second jaw base is fixedly secured to said elongated rod.

6. The clamp assembly of claim 4, wherein said second jaw base is movably secured to said elongated rod, such that a distance between said bases may be adjusted by moving either or both of said bases horizontally along said rod.

7. The clamp assembly of claim 4, wherein said first and second jaws are rotatably secured to said first and second bases, respectively, such that each of said jaws may be rotated about a vertical axis extending perpendicular to said elongated rod.

8. The clamp assembly of claim 7, wherein at least one of said first and second jaws may rotate up to 360 degrees about said vertical axis.

9. The clamp assembly of claim 4, wherein said first and second jaws each have a height that may be adjusted vertically along a vertical axis extending perpendicular to said elongated rod.

10. The clamp assembly of claim 9, wherein each of said first and second jaws include a terminal rod portion housed within a sleeve of each of said bases, such that each of said jaws may be moved vertically along said vertical axis of said assembly.

11. A clamp assembly for attachment to a patient's hip in knee arthroscopy, said assembly comprising:
a. a pair of opposing jaws secured to an elongated rod, each of said jaws configured to attach externally to a patient's hip;
b. a waypoint fixation mount secured to said rod and positioned between said pair of opposing jaws, said waypoint fixation mount designed for attachment to a computer assisted orientation system, and wherein said system is programmed to generate data corresponding to a position of said fixation mount for use in said knee arthroscopy, said computer assisted orientation system further programmed, through a series of algorithmic calculations, to establish the center of a femoral head of said patient prior to or during said knee arthroscopy.

12. The clamp assembly of claim 11, said pair of opposing jaws comprising a first jaw and a second jaw, and wherein said first jaw is secured to a base, said base movably secured to said elongated rod, such that said base, in combination with said first jaw, may be moved horizontally a desired distance away from said second jaw in order accommodate a width of said patient's hip.

13. The clamp assembly of claim 12, wherein said second jaw is secured to a base, said second jaw base secured to said elongated rod.

14. The clamp assembly of claim 12, wherein said first and second jaws are rotatably secured to said first and second bases, respectively, such that each of said jaws may be rotated about a vertical axis extending perpendicular to said elongated rod.

15. The clamp assembly of claim 14, wherein said first and second jaws each have a height that may be adjusted vertically along a vertical axis extending perpendicular to said elongated rod.

16. A method of establishing the center of the femoral head prior to or during knee arthroscopy comprising:
a. securing a clamp assembly externally about a patient's hips, said clamp assembly including:
i) a pair of opposing jaws secured to an elongated rod, each of said jaws configured to attach externally to a patient's hip; and
ii) a waypoint fixation mount secured to said rod and positioned between said pair of opposing jaws, said waypoint fixation mount designed for attachment to a computer assisted orientation system, and wherein said system is programmed to generate data corresponding to a position of said fixation mount for use in said knee arthroscopy; and
b. measuring the location of the center of the femoral head via said waypoint fixation mount and said computer assisted orientation system.

17. The method of claim 17, said pair of opposing jaws of said clamp assembly comprising a first jaw and a second jaw, and wherein said first jaw is secured to a base, said base movably secured to said elongated rod, such that said base, in combination with said first jaw, may be moved horizontally a desired distance away from said second jaw in order accommodate a width of said patient's hip.

18. The method of claim 17, wherein said second jaw of said clamp assembly is secured to a base, said second jaw base secured to said elongated rod.

19. The method of claim 17, wherein said first and second jaws of said clamp assembly are rotatably secured to said first and second bases, respectively, such that each of said jaws may be rotated about a vertical axis extending perpendicular to said elongated rod.

20. The method of claim 19, wherein said first and second jaws of said clamp assembly each have a height that may be adjusted vertically along a vertical axis extending perpendicular to said elongated rod.
